(19) United States
(12) Patent Application Publication
(10) Pub. No.: US 2015/0025074 A1
(43) Pub. Date: Jan. 22, 2015

(54) METHODS AND COMPOSITIONS FOR TREATING SOLID TUMORS AND OTHER MALIGNANCIES

(71) Applicants: Silvia Buonamici, Boston, MA (US); Marion Dorsch, Jamaica Plain, MA (US); Carlos Garcia-Echeverria, Saint Cloud (FR)

(72) Inventors: Silvia Buonamici, Boston, MA (US); Marion Dorsch, Jamaica Plain, MA (US); Carlos Garcia-Echeverria, Saint Cloud (FR)

(21) Appl. No.: 14/510,713

(22) Filed: Oct. 9, 2014

Related U.S. Application Data
(63) Continuation of application No. 13/509,857, filed on May 15, 2012.
(60) Provisional application No. 61/262,342, filed on Nov. 18, 2009.
(60) Provisional application No. 61/292,032, filed on Jan. 4, 2010.

(51) Int. Cl. A61K 31/5377; C07D 413/04; A61K 31/436

(52) U.S. Cl. CPC A61K 31/5377; A61K 31/436; C07D 413/04
USPC 514/235.5

(57) ABSTRACT

A combination of a kinase inhibitors of mTOR and downstream effector and a hedgehog pathway inhibitor for the treatment of cancer.

### BACKGROUND OF THE INVENTION

1. Field of the Invention

A combination of an mTOR inhibitor and a hedgehog pathway inhibitor for the treatment of solid tumors and hematological malignancies.

2. Related Background Art

The Hedgehog signaling pathway has been described in the art (see, e.g., Nybakken et al., Curr. Opin. Genet. Dev. 2002, 12:503-511; and Lum et al., Science 2003, 299: 2039-2045). Briefly, in the absence of hedgehog ligands, the transmembrane receptor, Patched (Ptch), binds to Smoothened (Smo) and blocks Smo's function. This inhibition is relieved in the presence of ligands, which allows Smo to initiate a signaling cascade that results in the release of transcription factors Glis from cytoplasmic proteins fused (Fu) and Suppressor of Fused (SuFu). In the inactive situation, SuFu prevents Glis from translocating to the nucleus. In the active situation, Fu inhibits SuFu and Glis are released. Gli proteins translocate into the nucleus and control target gene transcription.

Normally, Hh signaling is strictly controlled during cellular proliferation, differentiation and embryonic pattern formation. However, aberrant activity of the Hedgehog signaling pathway, due to mutations that constitutively activate the pathway, for instance, may have pathological consequences. By way of example, loss-of-function mutations of Patched are found in Gorlin's syndrome (a hereditary syndrome with high risk of skin and brain cancers, also known as Basal Cell Nevus Syndrome (BCNS)) and sporadic BCC and medulloblastoma; and gain-of-function mutations of Smo and Gli are linked to basal cell carcinoma, medulloblastoma and glioblastoma. Basal cell carcinoma (BCC) is the most common form of skin cancer, affecting more than 90,000 Americans each year.

Constitutive activation of Hh has been found to promote tumorigenesis in BCC, medulloblastoma (the most common childhood brain tumor), rhabdomyosarcoma, pancreatic cancer, small cell lung cancer, prostate cancer and breast cancer. Besides the roles in tumorigenesis, Hh signaling is also implicated in the metastasis of prostate cancer. Hh signaling may be involved in many additional types of tumor types and such links are expected to continue to be discovered; this is an area of active research in many cancer centers around the world.

Proliferation of these cancer cells requires Hh pathway activation, and blocking Hh signaling pathways often inhibits cancer cell proliferation. Indeed, Hh antagonist cyclopamine and anti-Gil1 siRNA can effectively block the proliferation of these cancer cells, and can reduce tumor size in Xenograft models, suggesting that Hh antagonists, alone or in combination with other agents, could provide new chemotherapeutic regimens for the treatment of these cancers. Hh antagonist cyclopamine has been shown to suppress the metastasis of prostate cancer in animal models.

Evidence that constitutive activation of Smo results in cancers (e.g., BCC), and that Smo may be oncogenic upon its release from inhibition by Ptch, suggests utility of Smo antagonists, alone or in combination, as therapeutic agents in the treatment of such disorders. (Stone et al. (1996) Nature 384; 129). Accordingly, molecules that modulate the activity of the Hedgehog signaling pathway, e.g., which modulate Smo activity, are therapeutically useful.

mTOR (mammalian target of rapamicin) is a kinase protein predominantly found in the cytoplasm of the cell. It acts as a central regulator of many biological processes related to cell proliferation, angiogenesis, and cell metabolism. mTOR exerts its effects primarily by turning on and off the cell's translational machinery, which includes the ribosomes, and is responsible for protein synthesis. mTOR is a key intracellular point of convergence for a number of cellular signaling pathways. mTOR performs its regulatory function in response to activating or inhibitory signals transmitted through these pathways, which are located upstream from mTOR in the cell. These diverse signaling pathways are activated by a variety of growth factors (including vascular endothelial growth factors (VEGFs), platelet-derived growth factor (PDGF), epidermal growth factor (EGF), insulin-like growth factor 1 (IGF-1)), hormones (estrogen, progesterone), and the presence or absence of nutrients (glucose, amino acids) or oxygen. One or more of these signaling pathways may be abnormally activated in patients with many different types of cancer, resulting in deregulated cell proliferation, tumor angiogenesis, and abnormal cell metabolism.

### BRIEF SUMMARY OF THE INVENTION

The invention provides a combination comprising a first agent that inhibits the hedgehog signaling pathway and a second agent that inhibits mTOR, ie the kinase activity of mTOR and its downstream effectors. In another aspect, the invention provides pharmaceutical compositions comprising a therapeutically effective amount of a first agent that inhibits hedgehog signaling pathway, a second agent that inhibits the kinase activity of mTOR and downstream effectors, and a pharmaceutically acceptable carrier.

In this regard, activation of the hedgehog pathway in human tissues can result in diseases such as psoriasis and specific types of cancer that include, but are not limited to, malignant lymphoma (LM), multiple myeloma (MM), cancers of the brain, muscle and skin, prostrate, medulloblastoma, pancreatic adenocarcinomas and small-cell lung carcinomas. Enhanced activation of the hedgehog signaling pathway contributes to the pathology and/or symptomology of a number of diseases. Accordingly, molecules that modulate the activity of the hedgehog signaling pathway are useful as therapeutic agents in the treatment of such diseases.

Furthermore, the present invention provides for the use of a therapeutically effective amount of a combination comprising a first agent that inhibits hedgehog signaling pathway and a second agent that inhibits the kinase activity of mTOR and downstream effectors, or pharmaceutically acceptable salts or pharmaceutical compositions thereof, in the manufacture of a medicament for treating a cell proliferative disorder, particularly cancer.

In the above compositions and methods for using the compositions of the invention, the first agent in the inventive composition may bind to Smo and the second agent in the inventive composition is an inhibitor of the kinase activity of mTOR.

In the above combinations, compositions and methods for using the compositions of the invention, the inventive composition may be administered to a system comprising cells or tissues. In some embodiments, the invention composition may be administered to a human patient or animal subject.

### BRIEF DESCRIPTION OF THE DRAWINGS

FIG. 1 shows the effect of a combination of Compound A and Compound 1 on Ptch+/−Hic+/− allograft medulloblastoma model and demonstrates by the reduction of tumor volume that the combination prevents or delays resistance to the antitumor activity of compound 1.

FIG. 2 shows in the Ptch+/−Hic+/− allograft medulloblastoma model the effect of a combination of Compound A and Compound 1 in terms of time to endpoint and shows the prevention or delay in resistance to the antitumor activity of compound 1.

### DETAILED DESCRIPTION OF THE INVENTION

The present invention is further exemplified, but not limited, by the following representative examples, which are intended to illustrate the invention and are not to be construed as being limitations thereon.

Compounds for Formula I—Smoothened Inhibitors

In one aspect, the present invention provides a compound of Formula I:

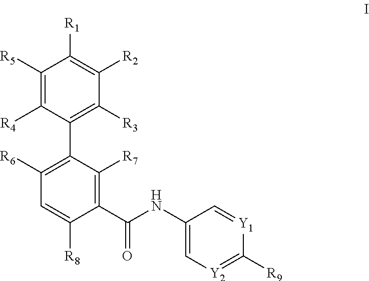

in which

Y₁ and Y₂ are independently selected from N and CR₁₀; wherein R₁₀ is selected from hydrogen, halo, C₁₋₆alkyl, halosubstituted-C₁₋₆alkyl, C₁₋₆alkoxy, halosubstituted-C₁₋₆alkoxy and —OXNR₁₀ₐR_{10b}; wherein R₁₀ₐ and R_{10b} are independently selected from hydrogen and C₁₋₆alkyl;

R₁ is selected from cyano, halo, C₁₋₆alkyl, halosubstituted-C₁₋₆alkyl, C₁₋₆alkoxy, halosubstituted-C₁₋₆alkoxy, C₆₋₁₀aryl, dimethyl-amino, C₁₋₆alkyl-sulfanyl and C₃₋₈heterocycloalkyl optionally substituted with up to 2 C₁₋₆alkyl radicals;

R₂ and R₅ are independently selected from hydrogen, cyano, halo, C₁₋₆alkyl, halosubstituted-C₁₋₆alkyl, C₁₋₆alkoxy, halosubstituted-C₃₋₆alkoxy and dimethylamino;

R₃ and R₄ are independently selected from hydrogen, halo, cyano, C₁₋₆alkyl, halosubstituted-C₁₋₆alkyl, C₁₋₆alkoxy and halosubstituted-C₁₋₆alkoxy; or either R₁ and R₂ or R₁ and R₅ together with the phenyl to which they are both attached form C₅₋₁₀heteroaryl;

R₆ and R₇ are independently selected from hydrogen. C₁₋₆alkyl, halosubstituted-C₁₋₆alkyl, C₁₋₆alkoxy and halosubstituted-C₁₋₆alkoxy; with the proviso that R₆ and R₇ are not both hydrogen

R₉ is selected from hydrogen, halo, C₁₋₆alkyl, halosubstituted-C₁₋₆alkyl, C₁₋₆alkoxy and halosubstituted-C₁₋₆alkoxy;

R₉ is selected from —S(O)₂R₁₁, —C(O)R₁₁, —OR₁₁, —NR₁₂ₐR_{12b} and —R₁₁; wherein R₁₁ is selected from aryl, heteroaryl, cycloalkyl and heterocycloalkyl; R₁₂ₐ and R_{12b} are independently selected from C₁₋₆alkyl and hydroxy-substituted-C₁₋₆alkyl;

wherein said aryl, heteroaryl, cycloalkyl and heterocycloalkyl of R₉ can be optionally substituted with 1 to 3 radicals independently selected from C₁₋₆alkyl, halosubstituted-C₁₋₆alkyl, C₁₋₆alkoxy, halosubstituted-C₁₋₆alkoxy, C₆₋₁₀aryl-C₀₋₄alkyl, C₅₋₁₀heteroaryl-C₀₋₄alkyl, C₃₋₁₂cycloalkyl and C₃₋₈heterocycloalkyl;

wherein said aryl-alkyl substituent of R₉ is optionally substituted with 1 to 3 radicals independently selected from halo, C₁₋₆alkyl, halosubstituted-C₁₋₆alkyl, C₁₋₆alkoxy, halosubstituted-C₁₋₆alkoxy and methyl-piperazinyl; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula 1 or a N-oxide derivative, individual isomers and mixture of isomers thereof; or a pharmaceutically acceptable salt thereof, in admixture with one or more suitable excipients.

Compounds of Formula 1 are hedgehog pathway inhibitors.

Preferred compounds of Formula 1 are selected from 4′-cyano-6-methyl-biphenyl-3-carboxylic acid [4-(morpholine-4-sulfonyl)-phenyl]-amide, 4′-cyano-6-methyl-biphenyl-3-carboxylic acid [6-(2,6-dimethyl-morpholin-4-yl)-pyridin-3-yl]-amide, 4′-Cyano-2-methyl-biphenyl-3-carboxylic acid (6-azepan-1-yl-pyridin-3-yl)-amide, 4′-Methoxy-2-methyl-biphenyl-3-carboxylic acid (6-azepan-1-yl-pyridin-3-yl)-amide, 4′-Methoxy-2-methyl-biphenyl-3-carboxylic acid (4-cyclohexyl-phenyl)-amide, 4′-Methoxy-2-methyl-biphenyl-3-carboxylic acid [6-(2-methyl-morpholin-4-yl)-pyridin-3-yl]-amide, 4′-Dimethylamino-2-methyl-biphenyl-3-carboxylic acid (4-cyclohexyl-phenyl)-amide, 4′-Dimethylamino-2-methyl-biphenyl-3-carboxylic acid (4-morpholin-4-yl-phenyl)-amide, 6-Chloro-4′-dimethylamino-biphenyl-3-carboxylic acid (6-[1,4]oxazepan-4-yl pyridin-3-yl)-amide, 6-Chloro-4′-dimethylamino-biphenyl-3-carboxylic acid (6-morpholin-4-yl-pyridin-3-yl)-amide, 6-Chloro-4-dimethylamino-biphenyl-3-carboxylic acid (6-azepan-1-yl-pyridin-3-yl)-amide, 6-Chloro-4′-methoxy-biphenyl-3-carboxylic acid [6-(2-methyl-morpholin-4-yl)-pyridin-3-yl]-amide, 6-Chloro-4′-methoxy-biphenyl-3-carboxylic acid (6-[1,4]oxazepan-4-yl-pyridin-3-yl)-amide, 6-Chloro-4′-methoxy-biphenyl-3-carboxylic acid (6-azepan-1-yl-pyridin-3-yl)-amide, 6-Chloro-4′-methoxy-biphenyl-3-carboxylic acid (6-morpholin-4-yl-pyridin-3-yl)-amide, 4′-Methoxy-6-methyl-biphenyl-3-carboxylic acid (6-morpholin-4-yl-pyridin-3-yl)-amide, 4′-Methoxy-6-methyl-biphenyl-3-carboxylic acid (6-[1,4]oxazepan-4-yl-pyridin-3-yl)-amide, 4′-Methoxy-6-methyl-biphenyl-3-carboxylic acid [6-(2-methyl-morpholin-4-yl)-pyridin-3yl]-amide, 4′-Dimethylamino-6-methyl-biphenyl-3-carboxylic acid [6-(2-methyl-morpholin-4-yl)-pyridin-3-yl]-amide, 4′-Dimethylamino-6-methyl-biphenyl-3-carboxylic acid (6-[1,4]oxazepan-4-yl-pyridin-3-yl)-amide, 4′-Dimethylamino-6-methyl-biphenyl-3-carboxylic acid (6-morpholin-4-yl-pyridin-3-yl)-amide, 4′-Methoxy-6-methyl-biphenyl-3-carboxylic acid (6-azepan-1-yl-pyridin-3-yl)-amide, 4′-Ethoxy-6-methyl-biphenyl-3-carboxylic acid (6-azepan-1-yl-pyridin-3-yl)-amide, 6-Methyl-4′-methylsulfanyl-biphenyl-3-carboxylic acid (6-azepan-1-yl-pyridin-3-yl)-amide, 4′-Dimethylamino-6-methyl-biphenyl-3-carboxylic acid (6-azepan-1-yl-pyridin-3-yl)-amide, 6-Methyl-[1,1′;4′,1″]terphenyl-3-carboxylic acid (6-azepan-1-yl-pyridin-3-yl)-amide, 3′-Chloro-6-methyl-biphenyl-3-carboxylic acid (6-azepan-1-yl-pyridin-3-yl)-amide, 2′,4′-Dichloro-6-methyl-biphenyl-3-carboxylic acid (6-azepan-1-yl-pyridin-3-yl)-amide, 2′-Chloro-6-methyl-biphenyl-3-carboxylic acid (6-azepan-1-yl-pyridin-3-yl)-amide, 3′-Chloro-6-methyl-biphenyl-3-carboxylic acid (6-azepan-1-yl-pyridin-3-yl)-amide, 3′,4′-Dichloro-6-methyl-biphenyl-3-carboxylic acid (6-azepan-1-yl-pyridin-3-yl)-amide, 3′-Chloro-6-methyl-4′-trifluoromethyl-biphenyl-3-carboxylic acid (6-azepan-1-yl-pyridin-3-yl)-amide, 6,4′-Dimethyl-biphenyl-3-carboxylic acid (6-azepan-1-yl-pyridin-3-yl)-amide, 4′-Ethyl-6-methyl-biphenyl-3-carboxylic acid (6-azepan-1-yl-pyridin-3-yl)-amide, 4′-tert-Butyl-6-methyl-biphenyl-3-carboxylic acid (6-azepan-1-yl-pyridin-3-yl)-amide, 6-Methyl-4′-propyl-biphenyl-3-carboxylic acid (6-azepan-1-yl-pyridin-3-yl)-amide, 4′-Isobutyl-6-methyl-biphenyl-3-carboxylic acid (6-azepan-1-yl-pyridin-3-yl)-amide, 4′-Isopropyl-6-methyl-biphenyl-3-carboxylic acid (6-azepan-1-yl-pyridin-3-yl)-amide, 6,2′,6′-Trimethyl-biphenyl-3-carboxylic acid (6-azepan-1-yl-pyridin-3-yl)-amide, 6,2′,3′-Trimethyl-biphenyl-3-carboxylic acid (6-azepan-1-yl-pyridin-3-yl)-amide, 6-Methyl-4′-trifluromethyl-biphenyl-3-carboxylic acid (6-azepan-1-yl-pyridin-3-yl)-amide, 6-Methyl-3′-trifluoromethyl-biphenyl-3-carboxylic acid (6-azepan-1-yl-pyridin-3 yl)-amide, 6-Methyl-3′,5′-bistrifluoromethyl-biphenyl-3-carboxylic acid (6-azepan-1-yl-pyridin-3-yl)-amide, 3′-Isopropoxy-6-methyl-biphenyl-3-carboxylic acid (6-azepan-1-yl-pyridin-3-yl)-amide, 3′-Ethoxy-6-methyl-biphenyl-3-carboxylic acid (6-azepan-1-yl-pyridin-3-yl)-amide, 2′,6′-Dimethoxy-6 methyl-biphenyl-3-carboxylic acid (6-azepan-1-yl-pyridin-3-yl)-amide, 6-Methyl-4′-trifluoromethoxy-biphenyl-3-carboxylic acid (6-azepan-1-yl-pyridin-3-yl)-amide, 6-Methyl-3′-trifluoromethoxy-biphenyl-3-carboxylic acid (6-azepan-1-yl-pyridin-3-yl)-amide, 6-Methyl-biphenyl-3-carboxylic acid (4-morpholin-4-yl-phenyl)-amide, 4′-Methoxy-6-methyl-biphenyl-3-carboxylic acid (4-morpholin-4-yl-phenyl)-amide, 3′-Methoxy-6-methyl-biphenyl-3-carboxylic acid (4-morpholin-4-yl-phenyl)-amide, 4′-(2-Dimethylamino-ethoxy)-6-methyl-biphenyl-3-carboxylic acid (4-morpholin-4-yl-phenyl)-amide. 3′-Dimethylamino-6-methyl-biphenyl-3-carboxylic acid (4-morpholin-4-yl-phenyl)-amide, 4′-Fluoro-6-methyl-biphenyl-3-carboxylic acid (4-morpholin-4-yl-phenyl)-amide, 3′-1-Fluoro-6-methyl-biphenyl-3-carboxylic acid (4-morpholin-4-yl-phenyl)-amide, 2′-Fluoro-6-methyl-biphenyl-3-carboxylic acid (4-morpholin-4-yl-phenyl)-amide, 4-Methyl-N-(4-morpholin-4-yl-phenyl)-3-quinoxalin-6-yl-benzamide, 6-Methyl-4′-(4-methyl-piperazin-1-yl)-biphenyl-3-carboxylic acid (4-morpholin-4-yl-phenyl)-amide, 2′-Cyano-6-methyl-biphenyl-3-carboxylic acid (4-morpholin-4-yl-phenyl)-amide, 3′-Cyano-6-methyl-biphenyl-3-carboxylic acid (4-morpholin-4-yl-phenyl)-amide, 4′-Cyano-6-methyl-biphenyl-3-carboxylic acid (6-[1,4]oxazepan-4-yl-pyridin-3-yl)-amide, 4′-Cyano-6-methyl-biphenyl-3-carboxylic acid (6-azepan-1-yl-pyridin-3-yl)-amide, 4′-Cyano-6-methyl-biphenyl-3-carboxylic acid [6-(2-methyl-morpholin-4-yl)-pyridin-3-yl]-amide, 4′-Cyano-6-methyl-biphenyl-3-carboxylic acid (3,4,5,6-tetrahydro-2H-[1,2]bipyridinyl-5′-yl)-amide, 4′-Cyano-6-methyl-biphenyl-3-carboxylic acid (6-morpholin-4-yl-pyridin-3-yl)amide, 4′-Cyano-6-methyl-biphenyl-3-carboxylic acid [6-(4-methyl-piperazin-1-yl)-pyridin-3-yl]-amide, 4′-Cyano-6-methyl-biphenyl-3-carboxylic acid (4-morpholin-4-yl-phenyl)-amide, 4′-Cyano-6-methyl-biphenyl-3-carboxylic acid (3-fluoro-4-morpholin-4-yl-phenyl)-amide, 4′-Cyano-6-methyl-biphenyl-3-carboxylic acid (3-chloro-4-morpholin-4-yl-phenyl)-amide, 4′-Cyano-6-methyl-biphenyl-3-carboxylic acid (3 bromo-4-morpholin-4-yl-phenyl)-amide, 4′-Cyano-6-methyl-biphenyl-3-carboxylic acid (3-methyl-4-morpholin-4-yl-phenyl)-amide, 4′-Cyan-6-methyl-biphenyl-3-carboxylic acid (4-morpholin-4-yl-3-trifluoromethyl-phenyl)-amide, 4′-Cyano-6-methyl-biphenyl-3-carboxylic acid (4-cyclohexyl-phenyl)-amide, 4′-Cyano-6-methyl-biphenyl-3-carboxylic acid biphenyl-4-ylamide, 4′-Cyano-6-methyl-biphenyl-3-carboxylic acid (4′-methoxy-biphenyl-4-yl)-amide, 4′-Cyano-6-methyl-biphenyl-3-carboxylic acid [4-(4-benzyl-piperazin-1-yl)-phenyl]-amide, 4′-Cyano-6-methyl-biphenyl-3-carboxylic acid [4-(piperidine-1-sulfonyl)-phenyl]-amide, 4′-Cyano-6-methyl-biphenyl-3-carboxylic acid (4-(pyrrolidine-1-sulfonyl)-phenyl-amide, 4′-Cyano-6-met oxy-biphenyl-3-carboxylic acid (6-azepan-1-yl-pyridin-3-yl)-amide, 4′-Cyano-2-methoxy-biphenyl-3-carboxylic acid (6-azepan-1-yl-pyridin-3-yl)-amide, 4′-Cyano-2-methyl-biphenyl-3-carboxylic acid (6-azepan-1-yl-pyridin-3-yl)-amide, 3′-Fluoro-4′-methoxy-6-methyl-biphenyl-3-carboxylic acid (6-azepan-1-yl-pyridin-3-yl)-amide, 4′-Isopropoxy-6-methyl-biphenyl-3-carboxylic acid (6-azepan-1-yl-pyridin-3-yl)-amide, 4′-Butoxy-6-methyl-biphenyl-3-carboxylic acid (6-azepan-1-yl-pyridin-3-yl)-amide. 3′-Chloro-4′-methoxy-6-methyl-biphenyl-3-carboxylic acid (6-azepan-1-yl-pyridin-3-yl)-amide, 4′-Methoxy-6,3′-dimethyl-biphenyl-3-carboxylic acid (6-azepan-1-yl-pyridin-3-yl)-amide, 4′-Cyano-2-methyl-biphenyl-3-carboxylic acid [4-(piperidine-1-sulfonyl)-phenyl]-amide, 4′-Cyano-6-fluoro-biphenyl-3-carboxylic acid [4-(piperidine-1-sulfonyl)-phenyl]-amide, 6-Bromo-4′-cyano-biphenyl-3-carboxylic acid [4-(piperidine-1-sulfonyl)-phenyl]-amide, 4′-Cyano-6-methyl-biphenyl-3-carboxylic acid [6-(4-benzyl-[1,4]diazepan-1-yl)-pyridin-3-yl]-amide, 4′-Cyano-6-methyl-biphenyl-3-carboxylic acid [6-(4-thiophen-3-ylmethyl-[1,4]diazepan-1-yl)-pyridin-3-yl]-amide, 4′-Cyano-2-methyl-biphenyl-3-carboxylic acid (6-(2,6-dimethyl-morpholin-4-yl)-pyridin-3 yl)-amide, 4-Methoxy-2-methy-biphenyl-3-carboxylic acid [6-(2,6-dimethyl-morpholin-4-yl)-pyridin-3-yl]-amide, 2-Methyl-4-trifluoromethyl-biphenyl-3-carboxylic acid [6 (2,6-dimethyl-morpholin-4-yl)-pyridin-3-yl]-amide, 2-Methyl-4′-trifluoromethoxy-biphenyl-3-carboxylic acid [6-(2,6-dimethyl-morpholin-yl)-pyridin-3-yl]-amide, 4′-Cyano-2-methyl-biphenyl-3-carboxylic acid [6-(2-methyl-morpholin-4-yl)-pyridin-3-yl]-amide, 4′-Cyano-2-fluoro-biphenyl-3-carboxylic acid [4 (piperidine-1-sulfonyl)-phenyl]-amide, 4′-Cyano-6-trifluormethyl-biphenyl-3-carboxylic acid [4-(piperidine-1-sulfonyl)-phenyl]-amide, 4′-Cyano-6-methyl-biphenyl-3-carboxylic acid [6-(4-pyridin-4-ylmethyl-[1,4]diazepan-1-yl)-pyridin-3-yl]-amide, 4′-Cyano-6-methyl-biphenyl-3-carboxylic acid [6-(4-pyridin-3-ylmethyl-[1,4]diazepan-1-yl)-pyridin-3-yl]-amide, 4′-Cyano-6-methyl-biphenyl-3-carboxylic acid {6-[4-(2,6-dimethoxy-benzyl)-[1,4]diazepan-1-yl]-pyridin-3 -yl}-amide, 4′-Cyano-6-methyl-biphenyl-3-carboxylic acid {6-(4-(2-ethoxy-benzyl)-[1,4]diazepan-1-yl)-pyridin-3-yl}-amide, 4′-Cyano-6-methyl-biphenyl-3-carboxylic acid (6-{4-[2-(4-methyl-piperazin-1-yl)-benzyl]-[1,4]diazepan-1-yl}-pyridin-3-yl)-amide, 4′-Cyano-6-methyl-biphenyl-3-carboxylic acid {6-[4-(4-methoxy-2,3-dimethyl-benzyl)-[1,4]diazepan-1-yl]-pyridin-3-yl}-amide, 4′-Cyano-6-methyl-biphenyl-3-carboxylic acid {6-[4-(2,3-dihydro-benzo[1,4]dioxin-6-ylmethyl)-[1,4]diazepan-1-yl]-pyridin-3-yl}-amide, 4′-Cyano-6-methyl-biphenyl-3-carboxylic acid [6-(4-pyridin-2-ylmethyl-[1,4]diazepan-1-yl)-pyridin-3-yl]-amide, 4′-Cyano-6-methyl-biphenyl-3-carboxylic acid [6-(4-benzo[1,3]dioxol-4-ylmethyl-[1,4]diazepan-1-yl)-pyridin-3-yl]-amide, 4′-Cyano-6-methyl-biphenyl-3-carboxylic acid {6-[4-(2-trifluoromethoxy-benzyl)-[1,4]diazepan-1-yl]-pyridin-3-yl}-amide, 4′-Cyano-6-methyl-biphenyl-3-carboxylic acid {6-[4-(2-dimethylamino-benzyl)[1,4]diazepan-1-yl]-pyridin-3-yl}-amide, 4′-Cyano-6-methyl-biphenyl-3-carboxylic acid {6-[4-(2-chloro-5-trifluoromethyl-benzyl)-[1,4]diazepan-1-yl]-pyridin-3-yl}-amide, 4′-Cyano-6-methyl-biphenyl-3-carboxylic acid {6-[4-(2,3-difluoro-benzyl)-[1,4]diazepan-1-yl]-pyridin-3-yl}-amide, 4-Cyano-6-methyl-biphenyl-3-carboxylic acid {6-[4-(2-chloro-4-fluoro-benzyl)-[1,4]diazepan-1-yl]-pyridin-3-yl}-amide, 4′-Cyano-6-methyl-biphenyl-3-carboxylic acid {6-[4-(2,6-difluoro-benzyl), [1,4]diazepan-1-yl]-pyridin-3-yl}-amide, 2-Chloro-4′-cyano-biphenyl-3-carboxylic acid [4-(piperidine-1-sulfonyl)-phenyl]-amide, 4′-Cyano-6-trifluoromethyl-biphenyl-3-carboxylic acid [6-(2,6-dimethyl-morpholin-4-yl)-pyridin-3-yl]-amide, 2-Chloro-4′-cyano-biphenyl-3-carboxylic acid [6-(2,6-dimethyl-morpholin-4-yl)pyridin-3-yl]-amide, 4′-Cyano-6-ethyl-biphenyl-3-carboxylic acid [6-(2,6-dimethyl-morpholin-4-yl)-pyridin-3-yl]-amide, 4′-Cyano-6-methyl-biphenyl-3-carboxylic acid {6-[4-(3-fluoro-benzyl)-piperazin-1-yl]-pyridin-3-yl}-amide, 4′-Cyano-6-methyl-biphenyl-3-carboxylic acid {6-[4-(2-trifluoromethoxy-benzyl)piperazin-1-yl]-pyridin-3 yl}-amide, 4′-Cyano-6-methyl-biphenyl-3-carboxylic acid {6-[4-(3-chloro-benzyl)-piperazin-1-yl]-pyridin-3-yl}-amide, 4′-Cyano-6-methyl-biphenyl-3-carboxylic acid {6-[4-(4-isobutyl-benzyl)-piperazin-1-yl]-pyridin-3-yl}-amide, 4′-Cyano-6-methyl-biphenyl-3-carboxylic acid {6-[4-(4-tert-butyl-benzyl)-piperazin-1-yl]-pyridin-3-yl}-amide, 4′-Cyano-6-methyl-biphenyl-3-carboxylic acid {6-[4-(7-methoxy-benzo[1,3]dioxol-5-ylmethyl)-piperazin-1-yl]-pyridin-3-yl}-amide, 4′-Cyano-6-methyl-biphenyl-3-carboxylic acid [6-(4-benzyl-piperazin-1-yl)-pyridin-3-yl]-amide, 4′-Cyano-6-methyl-biphenyl-3-carboxylic acid [6-(4-pyridin-3-ylmethyl-piperazin-1-yl)-pyridin-3-yl]-amide, 4′-Cyano-6-methyl-biphenyl-3-carboxylic acid {6-[4-(4-difluoromethoxy-benzyl)-piperazin-1-yl]-pyridin-3-yl}-amide, 4′-Cyano-6-methyl-biphenyl-3-carboxylic acid {6-[4-(4-cyano-benzyl)-piperazin-1-yl]-pyridin-3-yl}-amide, 4′-Cyano-6-methyl-biphenyl-3-carboxylic acid [6-(4-quinolin-5-ylmethyl-piperazin-1-yl)-pyridin-3-yl]-amide, 4′-Cyano-6-methyl-biphenyl-3-carboxylic acid [6-(4-pyridin-4-ylmethyl-piperazin-1-yl)-pyridin-3-yl]-amide, 4′-Cyano-6-methyl-biphenyl-3-carboxylic acid [6-(4-pyridin-2-ylmethyl-piperazin-1-yl)-pyridin-3-yl]-amide, 4′-Cyano-6-methyl-biphenyl-3-carboxylic acid {6-[4-(4-imidazol-1-yl-benzyl)-piperazin-1-yl]-pyridin-3-yl}-amide, 4′-Cyano-6-methyl-biphenyl-3-carboxylic acid {6-[4-(3-cyano-benzyl)-piperazin-1-yl]-pyridin-3-yl}-amide, 4′-Cyano-6-methyl-biphenyl-3-carboxylic acid [6-(4-isoquinolin-5-ylmethyl-piperazin-1-yl)-pyridin-3-yl]-amide, (R)-2-methyl-N-(6-(2-methylmorpholino)pyridin-3-yl)-4′-(trifluoromethoxy)biphenyl-3-carboxamide, 4′-cyano-2-methyl-N-(6-sulfonylmorpholinopyridin-3-yl)biphenyl-3-carboxamide, (S)-4′-cyano-2-methyl-N-(6-(2-methylmorpholino)pyridin-3-yl)biphenyl-3-carboxamide, (R)-6-chloro-N-(6-(2-methyl morpholino)pyridin-3-yl)-4′-(trifluoromethoxy)biphenyl-3-carboxamide, 4′-cyano-2-methyl-N-(6-sulfinylmorpholinopyridin-3-yl)biphenyl-3-carboxamide, 4′-cyano-N-(6-(diisobutylamino)pyridin-3-yl)-2-methylbiphenyl-3-carboxamide, 4′-cyan-N-(2-((2S,6R)-2,6-dimethylmorpholino)pyrimidin-5-yl)-2-methylbiphenyl-3-carboxamide, N-(2-((2S,6R)-2,6-dimethylmorpholino)pyrimidin-5-yl)-2-methyl-4′-(trifluoromethyl)biphenyl-3-carboxamide, N-(2-((2S,6R)-2,6-dimethylmorpholino)pyrimidin-5-yl)-2-methyl-4′-trifluoromethoxy)biphenyl-3-carboxamide, N-2-(bis(2-hydroxyethyl)amino)pyrimidin-5-yl)-2-methyl-4′-(trifluoromethoxy)biphenyl-3-carboxamide, 2-methyl-N-(6-(tetrahydro-2H-pyran-4-yloxy)pyridin-3-yl)-4′-(trifluoromethoxy)biphenyl-3-carboxamide, N-(5-chloro-6-((2S,6R)-2,6-dimethylmorpholino-3-yl)-2-methy-4′-(trifluoromethoxy)biphenyl-3-carboxamide, N-((2R,6S)-2,6-dimethyltetrahydro-2H-pyran-4-yl)pyridin-3-yl)-2-methyl-4′-(trifluoromethoxy)biphenyl-3-carboxamide, N-(6-(4-ethylpiperazine-1-carbonyl)pyridin-3-yl-2-methyl-4′-(trifluoromethoxy)biphenyl-3-carboxamide, 2-methyl-N-(6-(2-oxopiperazin-1-yl)pyridin-3-yl)-4′-(trifluoromethoxy)biphenyl-3-carboxamide, 2-methyl-N-(6(1-(pyridin-4-ylmethyl)piperidin-4-yl)pyridin-3-yl)-4′-(trifluoromethoxy)biphenyl-3-carboxamide, 2-methyl-N-(6 (2-oxo-4-(pyridin-4-ylmethyl)piperazin-1-ylpyridin-3-yl)-4′-(trifluoromethoxy)biphenyl-3-carboxamide, 2-methyl-N-(6-(pyridin-4-ylmethyl)piperidin-3-yl)pyridin-3-yl)-4′-(trifluoromethoxy)biphenyl-3-carboxamide, N-(6-(1-ethylpiperidin-3-yl)pyridin-3-yl)-2-methyl-4′-(trifluoromethoxy)biphenyl-3-carboxamide and N-(6-((2R,6S)-2,6-dimethylmorpholino)pyridin-3-yl)-2-methyl-4′-(trifluoromethoxy)biphenyl-3-carboxamide and 2-Methyl-4′-trifluoromethoxy-biphenyl-3-carboxylic acid [6-(cis-2,6-dimethyl-morpholin-4-yl)-pyridin-3-yl]-amide, (also identified as Compound 1 in this document), which has the formula:

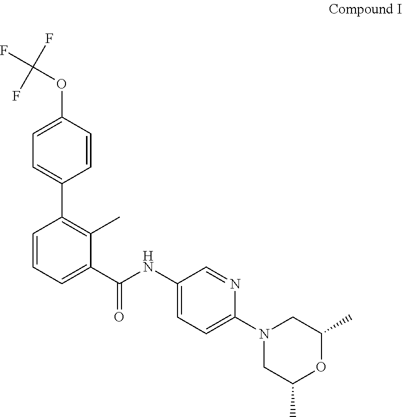 [SMILES: Cc1c(C(=O)Nc2ccc(N3C[C@@H](C)O[C@@H](C)C3)nc2)cccc1-c1ccc(OC(F)(F)F)cc1]

The above compounds of Formula 1 are further described in WO 2007/131201.

Compounds of Formula II—Smoothened Inhibitors

The present invention relates to a compounds of the formula (II):

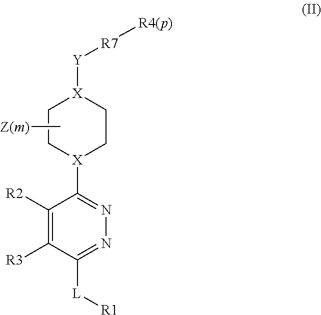

and pharmaceutically acceptable salts thereof, wherein

R1 is a C₆₋₁₄ aryl group, or a 5-14 membered heteroaryl group which may be unsubstituted or substituted;

R2 and R3 are independently C₁₋₈alkyl, C₁₋₈alkylOH, or R2 and R3 form a fused C₃₋₁₄ cycloalkyl group;

L is a bond, C₁₋₈ alkylene, —C(O)O—, —C(O)NR9-, —C₁₋₈ alkylOH—, —C₁₋₈, haloalkyl-, —C(O)—, —NH— or —O—;

X and W are independently N or CRS, and at least one of X or W is N;

R7 is a C₆₋₁₄ aryl group, a 5-14 membered heteroaryl group, or a 3-14 membered cycloheteroalkyl group;

R4 is C₁₋₈ alkyl, C₂₋₈ alkenyl, C₃₋₁₄ cycloalkyl, a C₃₋₁₄ aryl group, a 5-14 membered heteroaryl group, a 3-14 membered cycloheteroalkyl group, C₁₋₈ alkoxy, halo, NR6R8, C(O)OR6, C(O)NR6R8, C₁₋₈haloalkyl, formyl, carbalkoxy, C₁₋₈alkylOH, C(O)R6, SO₂CR6, C(O)NHC₁₋₈alkylR6, NR6R8, SO₂NR6R8, OCF₃, NHC(O)R6, CH₂OC(O)NR6R8, CH₂NR6R8, NHC(O)OR6, NHC(O)NR6R8, CH₂NHSO₂R6, CH₂NHC(O)OR6, OC(O)R6, or NHC(O)R6, which may be substituted or unsubstituted;

Z is C₁₋₈ alkyl, CN, OH, or halogen;

m and p are independently 0-3;

Y is a bond, C₁₋₈ alkylene, —C(O)—, —C(O)O—, —CH(OH)—, or —C(O)NR10;

R5 is H, halogen, CN, lower alkyl, OH, OCH₃ or OCF₃;

Wherein R1 may be substituted by one or more of C₁₋₄ alkyl, a C₁₋₁₄ aryl group, C₁₋₈ haloalkyl, C₁₋₈ alkoxy, halo, NH₂, CN, OCF₃, OH, C(O)NR6R8, C(O)R6, NR6R8, NHC(O)R6, SO₂R6, SO₂NR6R8;

R9 and R10 are independently C₁₋₈ alkyl or H;

R6 and R8 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈haloalkyl, C₁₋₈alkylOH, C₁₋₈alkoxy, or two R6 on one atom can form a heteroatom containing ring; and

Wherein R4, R6, and R8 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃, —OCF₃, —OH, —NH₂.

In another embodiment, the present invention includes compounds of formula (II) wherein R7 is

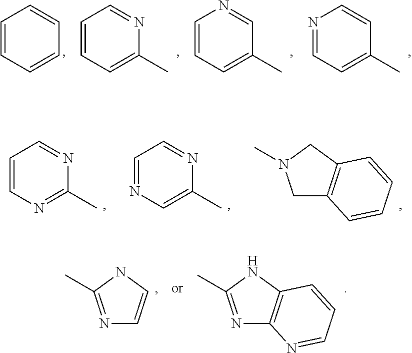

In another embodiment, the present invention includes compounds of formula (II) according to claim 1 wherein R1 is

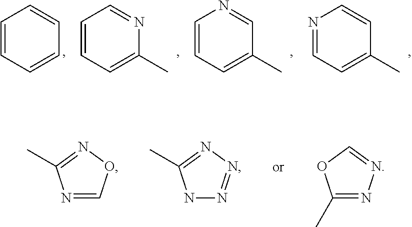

In another embodiment, the present invention includes compounds of formula (II) wherein R7 is

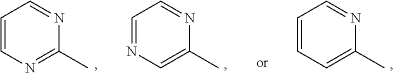

and

### R1 is

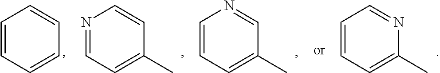

In yet another embodiment, the presets invention includes compounds of formula (II) wherein R4 is C(O)OC₁₋₈ alkyl, CF₃, C(O)OR6, C(O)NR6R8, C₁₋₈ haloalkyl, C₁₋₈ alkylOH, C(O)R6, SO₂R6, C(O)NHC₁₋₈alkylR6, C(CH₃)(CH₃)(OH), C(O)CH₃, C(CH₂)CH₃, or C(CH₃)(CH₂OH)OH; and

R6 and R8 are independently H, C₁₋₈ alkyl, C₁₋₈ alkenyl, C₃₋₁₄ cycloalkyl, a C₆₋₁₄ aryl group, a 5-14 membered heteroaryl group, or a 3-14 membered cycloheteroalkyl group.

In another embodiment, the present invention includes compounds of formula (II) wherein R4 is

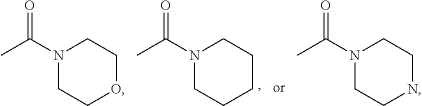

which may be unsubstituted or substituted.

In another embodiment, the present invention includes compounds of formula (II) wherein R2 and R3 are C₁₋₈ alkyl.

In a still further embodiment, the present invention includes compounds of formula (II) wherein R2 and R3 are CH₃.

In another embodiment, the present invention includes compounds of formula (II) wherein L, is —O—, —NH—, —C(O)—, —CH(OH)—, —CH₂—, —CF₂—, —CHF—, —COH—, or a bond. In another embodiment, the present invention includes compounds of formula (I) wherein L is —CH₂—. In another embodiment, the present invention includes compounds of formula (I) wherein both X are N, and Z is CH₃.

In another embodiment, the present invention includes a compound of formula (IIa):

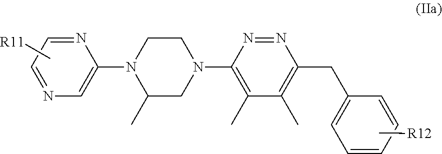

and pharmaceutically acceptable salts thereof, wherein

R11 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo, NR13R14, C(O)OR13, C(O)NR13R14, C₁₋₈haloalkyl, formyl, carbalkoxy, C₁₋₈alkylOH, C(O)R13, SO₂R13, C(O)NHC₁₋₈alkylR13, NR13R14, SO₂NR13R14, OCF₃, NHC(O)R13, CH₂OC(O)NR13R14, CH₂NR13R14, NHC(O)OR13, NHC(O)NR13R14, CH₂NHSO₂R13, CH₂NHC(O)OR13, OC(O)R13, or NHC(O)R13, which may be substituted or unsubstituted;

R12 is H, C₁₋₈ alkyl, a C₆₋₁₄ aryl group, C₁₋₈ haloalkyl, C₁₋₈ alkoxy, halo, NH₂, CN, OCF₃, OH, C(O)NR13R14, C(O)R13, NR13R14, NHC(O)R13, SO₂R13, SO₂NR13R14;

R13 and R14 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ haloalkyl, C₁₋₈ alkylOH, C₁₋₈ alkoxy. or R13 and R14 on one atom can form a heteroatom containing ring; and

Wherein R11, R13, and R14 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃, —OCF₃, —OH, —NH₂.

Compounds of Formula II and IIa are further described in the contents of U.S. patent application Ser. No. 12/503,565, which has counterpart International Application No. PCT/EP09/059138.

A preferred compound of formula (II) is 2-[(R)-4-(6-Benzyl-4,5-dimethyl-pyridazin-3-yl)-2-methyl-3,4,5,6-tetrahydro-2H-[1,2′]bipyrazinyl-5′-yl]-propan-2-ol, (also identified as Compound B in this document), of the below formula:

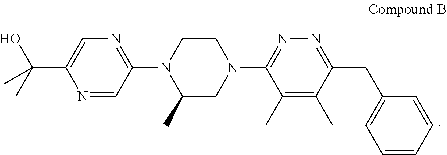 [SMILES: Cc1c(Cc2ccccc2)nnc(N2CCN(c3cnc(C(C)(C)O)cn3)[C@H](C)C2)c1C]

2-[(R)-4-(6-Benzyl-4,5-dimethyl-pyridazin-3-yl)-2-methyl-3,4,5,6-tetrahydro-2H-[1,2′]bipyrazinyl-5′-yl]-propan-2-ol can be made according to Scheme 1

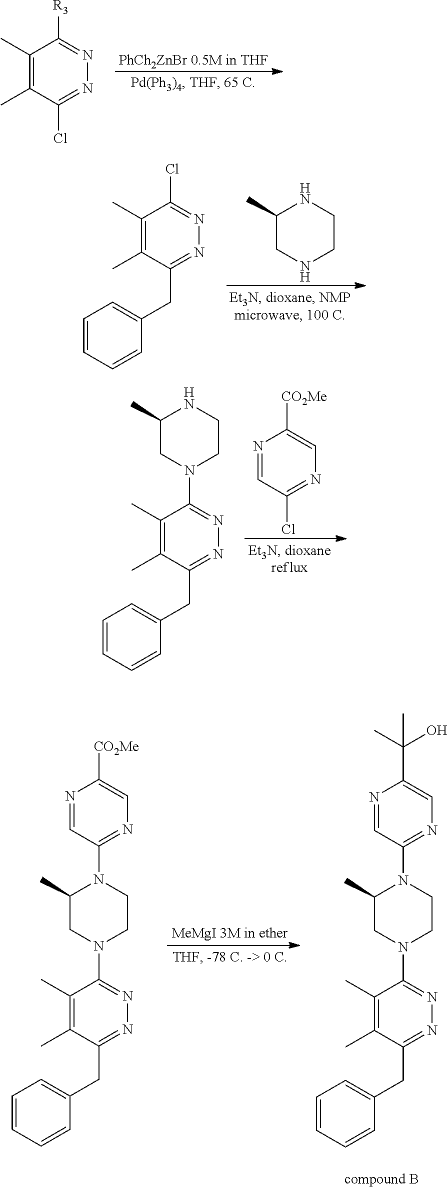

### First Step:

A mixture of 4,5-dimethyl-1,4-dichloro-pyridazine (10 g, 56.5 mmol), tetrakis(triphenylphosphine)palladium(0) (3.3 g, 2.80 mmol) and THF (200 mL) is degassed and then benzylzinc bromide (147 mL, 0.5 M in THF, 73.40 mmol) is added. The reaction solution is heated to 65° C. overnight. Solvent is removed. Water is added and the water layer is extracted with EtOAc. The organic layer is concentrated to afford a crude product that is purified by chromatography on silica gel (EtOAc/Heptane: 0%˜50%) to give 3-benzyl-6-chloro-4,5-dimethyl-pyridazine. 9.5 g, 67%).

### Second Step:

3-Chloro-4,5-dimethyl-6-((R)-3-methyl-piperazin-yl)-pyridazine (400 mg, 1.66 mmol, 1 eq) is added to a solution of benzylzinc bromide (12.3 mL 0.5 M in THF, 6.64 mmol, 4 eq) and tetrakis(triphenylphosphine)palladium (100 mg, 0.08 mmol, 0.05 eq) in a microwave vial. The vial is sealed and irradiated in the microwave at 100° C. (high absorption setting) for 40 min. The reaction mixture is concentrated and purified by silica gel chromatography (5-20% EtOAc/heptane) to 3-benzyl-4,5-dimethyl-6-((R)-3-methyl-piperazin-1-yl)-pyridazine (324 mg, 66%).

### Third Step:

A mixture of the above compound (6.0 g, 20.27 mmol), 5-chloropyrazine-2-carboxylic acid methyl ester (5.3 g, 30.30 mmol), Et₃N (6.2 g, 60.60 mmol) and dioxane (100 mL) is heated to reflux overnight. Solvent is removed. Saturated NH₄Cl solution is added and extracted with EtOAc. The organic layer is concentrated to afford the crude product that is purified by chromatography on silica gel (EtOAc/heptane: 50%-100%) to (R)-4-(6-benzyl-4,5-dimethyl-pyridazin-3-yl)-2-methyl-3,4,5,6-tetrahydro-2H-[1,2′]bipyrazinyl-5′-carboxylic acid methyl ester (6.6 g, 76%) as a yellow solid.

### Final Step:

To a solution of (R)-4-(6-benzyl-4,5-dimethyl-pyridazin-3-yl)-2-methyl-3,4,5,6-tetrahydro-2H-[1,2′]bipyrazinyl-5′-carboxylic acid methyl ester (840 mg, 1.85 mmol) in THF (12 mL) is added methyl magnesium bromide (5 ml, 15 mmol, 3 M in ether) at −78° C. The reaction mixture is stirred at 0° C. for 2 h then diluted with DCM and washed with NH₄Cl and water. The combined organic layers are washed with water, brine, dried over Na₂SO₄, filtered and concentrated down. Purification by HPLC of the crude product with acetontrile in water (from 10% to 95% with 3% 1-propanol) at 220 nm wavelength detection provides the desired compound B (400 mg, 50%) next to small amounts of the corresponding methyl ketone. The solvents are removed with a lyophilizer to provide the products as white powders.

mTOR Inhibitors

Exemplary mTOR inhibitors which may be used to practice the invention, include the following. mTOR allosteric inhibitors active against the mTORC1 complex, such as Sirolimus (AY-22989, Wyeth), Everolimus (RAD001, Novartis), Temsirolimus (CCI-779, Wyeth) and Deferolimus (AP-235731MK-8669, Ariad/Merck & Co). ATP competitive mTOR inhibitors active against the mTORC1 and mTORC2 complexes, such as AZD-8055 (AstraZeneca), Ku-0063794 (AstraZeneca), OSI-027 (OSI Pharmaceuticals) and WYE-125132 (Wyeth). Other mTOR inhibitors useful with the present invention include those disclosed in US Patent Application Publication Nos. 2008/0194546 and 2008/0081809, the contents of both being incorporated by reference herein. Additional mTOR ATP-competitive inhibitors useful with the present invention include, INK-128 (Intellikine) and EX2044, EX3855 and EX7518 (Exelisis).

Everolimus, which is compound A. has the chemical name ((1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclol-[30.3.1.04.9]hexatriaconta-16,24,26,28-tetraene-2,3,10,14,20-pentaone.) Everolimus and its are described in U.S. Pat. No. 5,665,772, at column 1, line 39 to column 3, line 11, which is incorporated by reference herein.

### DEFINITIONS

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl above where one or more of the ring members is a heteroatom. For example C₅₋₁₀heteroaryl is a minimum of 5 members as indicated by the carbon atoms but that these carbon atoms can be replaced by a heteroatom. Consequently, C₅₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N—, —NR—, —C(O)—, —S—, —S(O)— or —S(O)—, wherein R is hydrogen, C₁₋₄alkyl or a nitrogen protecting group. For example, C₃₋₈heterocycloalkyl as used in this application to describe compounds of the invention includes morpholino, pyrrolidinyl, pyrrolidinyl-2-one, piperazinyl, piperidinyl, piperidinylone, 1,4-dioxa-8-aza-spiro[4.5]dec-8-yl, thiomorpholino, sulfanomorpholino, sulfonomorpholino, etc.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazoliclinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetrahydroisoquinolinyl, isoquinolinyl, benzimidazolyl, benzopyranyl, indolizinyl, benzofuryl, chromonyl, coumarinyl, enzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl) and the like.

Exemplary tricyclic heterocyclic groups include carbazolyl, benzidolyl, phenanthrolinyl, acridinyl, phenanthridinyl, xanthenyl and the like.

“Halogen” (or halo) preferably represents chloro or fluoro, but may also be bromo or iodo.

The term “alkoxy” refers to an alkyl group as defined herein, connected to the main chain by an oxygen atom. Examples include but are not limited to methoxy, ethoxy, and the like.

The term “carbalkoxy” refers to an alkoxycarbonyl group, where the attachment to the main chain is through the carbonyl group (C(O)). Examples include but are not limited to methoxy carbonyl, ethoxy carbonyl, and the like.

As used herein, “oxo” refers to a double-bonded oxygen (i.e., =0). It is also to be understood that the terminology C(O) refers to a —C═O group, whether it be ketone, aldehyde or acid or acid derivative. Similarly, S(O) refers to a —S═O group.

The term “alkylene” as used herein refers to a straight or branched chain consisting solely of carbon and hydrogen. Examples of “alkylene” groups include methylene, ethylene, propylene, butylene, pentylene, and 3-methypentylene.

The term “alkenylene” as used herein refers to a straight or branched chain consisting solely of carbon and hydrogen, containing at least one carbon-carbon double bond. Examples of“alkenylene” groups include ethenylene, propenylene, butenylene, 3,3,-dimethylbut-1-enylene, 3-methylbut-1-enylene, pentenylene, 3-methylpentenylene, and butadiene.

As used herein, the term “sulfanyl” refers to a thio group.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc.

The term “substituted” is intended to describe moieties having substituents replacing a hydrogen on one or more atoms, e.g. C, O or N, of a molecule. Such substituents can include, for example, oxo, alkyl, alkoxy, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety, and any combination thereof.

Pharmaceutically acceptable salts of any acidic compounds of the invention are salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris-(hydroxymethyl)-methylammonium salts.

Similarly acid addition salts, such as of mineral acids, organic carboxylic, and organic sulfonic acids e.g., hydrochloric acid, methansulfonic acid, maleic acid, are possible provided a basic group, such as amino or pyridyl, constitutes part of the structure.

The compounds used with the methods of the invention, depending on the nature of the substituents, possess one or more asymmetric carbon atoms, and therefore exist as racemates and the (R) and (S) enantiomers thereof, and both enantiomers fall within the scope of the present invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, a chemical compound, or a combination of two or more substances. Unless otherwise specified, the terms “agent”, “substance”, and “compound” can be used interchangeably.

As used herein, “contacting” has its normal meaning and refers to combining two or more molecules (e.g., a small molecule organic compound and a polypeptide) or combining molecules and cells (e.g., a compound and a cell). Contacting can occur in vitro, e.g., combining two or more agents or combining a compound and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “hedgehog” is used to refer generically to any member of the hedgehog family, including sonic, indian, desert and tiggy winkle. The term may be used to indicate protein or gene. The term is also used to describe homolog/ortholog sequences in different animal species.

The terms “hedgehog (Hh) signaling pathway” and “hedgehog (Hh) signaling” are used interchangeably and refer to the chain of events normally mediated by various members of the signaling cascade such as hedgehog, patched (Ptch). smoothened (Smo), and Gli. The hedgehog pathway can be activated even in the absence of a hedgehog protein by activating a downstream component. For example, overexpression of Smo will activate the pathway in the absence of hedgehog.

Hh signaling components or members of Hh signaling pathway refer to gene products that participate in the Hh signaling pathway. An Hh signaling component frequently affects the transmission of the Hh signal in cells/tissues, typically resulting in changes in degree of downstream gene expression level and/or phenotypic changes. Hh signaling components, depending on their biological function and effects on the final outcome of the downstream gene activation/expression, may be divided into positive and negative regulators. A positive regulator is an Hh signaling component that positively affects the transmission of the Hh signal, i.e., stimulates downstream biological events when Hh is present. Examples include hedgehog, Smo, and Gli. A negative regulator is an Hh signaling component that negatively affects the transmission of the Hh signal, i.e., inhibits downstream biological events when Hh is present Examples include (but are not limited to) Ptch and SuFu. Smo is an essential component of the Hh signaling pathway.

Hedgehog signaling antagonists, antagonists of Hh signaling or inhibitors of Hh signaling pathway refer to agents that inhibit the bioactivity of a positive Hh signaling component (such as hedgehog, Ptch, or Gli) or down-regulate the expression of the Hh signaling component. They also include agents which up-regulate a negative regulator of Hh signaling component. A hedgehog signaling antagonist may be directed to a protein encoded by any of the genes in the hedgehog pathway, including (but not limited to) sonic, indian or desert hedgehog, smoothened, ptch-1, ptch-2, gli-1, gli-2, gli-3, etc.

The terms “inhibit,” “inhibiting” or “inhibition,” in the context of modulation of enzymatic activities, inhibition relates to reversible suppression or reduction of an enzymatic activity including competitive, uncompetitive, and noncompetitive inhibition. This can be experimentally distinguished by the effects of the inhibitor on the reaction kinetics of the enzyme, which may be analyzed in terms of the basic Michaelis-Menten rate equation. Competitive inhibition occurs when the inhibitor can combine with the free enzyme in such a way that it competes with the normal substrate for binding at the active site. A competitive inhibitor reacts reversibly with the enzyme to form an enzyme-inhibitor complex [EI], analogous to the enzyme-substrate complex.

The term “subject” includes mammals, especially humans. It also encompasses other non-human animals such as cows, horses, sheep, pigs, cats, dogs, mice, rats, rabbits, guinea pigs, monkeys. The term “patient” refers to a human patient.

The term “treating” includes the administration of compounds or agents to prevent or delay the onset of the symptoms, complications, or biochemical indicia of a disease, alleviating the symptoms or arresting or inhibiting further development of the disease, condition, or disorder. Treatment can include therapeutic suppression or alleviation of symptoms alter the manifestation of the disease.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopoeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Cancer”, as used herein, includes solid mammalian tumors as well as hematological malignancies. “Solid mammalian tumors” include cancers of the head and neck, lung, including small-cell lung and non-small-cell lung mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, muscle, central nervous system including brain, medulloblastoma, basal cell carcinoma, pancreas; sarcomas of the soft tissue and bone; and melanoma of cutaneous and intraocular origin. “Hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers.

Cancers which are particularly amenable to treatment by the methods of the invention include but are not limited to gliomas, medulloblastomas (e.g., cerebellar medulloblastomas), pericytoma, primitive neuroectodermal tumors (PNETS), basal cell carcinoma (BCC), small cell lung cancers, large cell lung cancers, tumors of the gastrointestinal tract, rhabdomyosarcomas, breast cancer, soft tissue sarcomas, pancreatic tumors, bladder tumors and prostate tumors.

“Hedgehog-related disorder(s)” as used herein includes disorders associated with disruption or aberrance of the Hedgehog pathway, as well as disorders associated with normal but undesired growth states relating to activation of the Hedgehog pathway. “Hedgehog-related disorder(s)” include but are not limited to tumor formation, cancer, neoplasia, malignant hyperproliferative disorders, and non-malignant hyperproliferative disorders. “Hedgehog-related disorder(s)” also include benign prostate hyperplasia, psoriasis, wet macular degeneration, osteopetrosis and unwanted hair growth. As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, central nervous system including brain; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated. Cancers which are particularly amenable to treatment by the methods of the invention include but are not limited to gliomas, medulloblastomas (e.g., cerebellar medulloblastomas, pericytoma, primitive neuroectodermal tumors (PNETS), basal cell carcinoma (BCC), small cell lung cancers, large cell lung cancers, tumors of the gastrointestinal tract, rhabdomyosarcomas, breast cancer, soft tissue sarcomas, pancreatic tumors, bladder tumors and prostate tumors.

As used herein, “sensitive tumors” means tumors (e.g., medulloblastomas) which, due to hedgehog pathway activation, respond to treatment with a smoothened inhibitor anti-cancer regimen.

As used herein, “resistant tumors” means formerly sensitive tumors (e.g., medulloblastomas) which, in the continuos presence of a smo inhibitor, either have regrown after shrinking due to treatment, or have reappeared after being temporarily eliminated due to treatment. Resistant tumors show a decreased sensitivity or no response to smoothened inhibition. Successful treatment of resistant tumors can engender, e.g., increased sensitivity of a tumor cell to novel or previously attempted anti-cancer regimen and/or chemotherapeutic agents, and can result in, e.g., subsequent tumor cell death and prevention from metastasis.

As used herein, the term “malignant hypeproliferative disorder(s)” includes but is not limited to cancers, neuronal proliferative disorders, bone marrow proliferative diseases and leukemias.

As used herein, the term “non-malignant hyperproliferative disorder(s)” includes but is not limited to non-malignant and non-neoplastic proliferative disorders, such as smooth muscle hyperplasia in blood vessels, cutaneous scarring, and pulmonary fibrosis.

Pharmacology and Utility

The combination of the present invention may be used for treating a variety of cancers. In one embodiment, the invention provides an agent that inhibits the hedgehog signaling pathway in combination with an agent that inhibits the kinase activity of mTOR and downstream effectors.

Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated. Cancers which are particularly amenable to treatment by the compounds and methods of the invention include but are not limited to gliomas, medulloblastomas, primitive neuroectodermal tumors (PNETS), basal cell carcinoma (BCC), small cell lung cancers, large cell lung cancers, tumors of the gastrointestinal tract, rhabdomyosarcomas, soft tissue sarcomas, pancreatic tumors, bladder tumors and prostate tumors. As used herein, the term “malignant hyperproliferative disorder(s)” includes but is not limited to cancers, neuronal proliferative disorders, bone marrow proliferative diseases and leukemias. As used herein, the term “non-malignant hyperproliferative disorder(s)” includes but is not limited to non-malignant and non-neoplastic proliferative disorders, such as smooth muscle hyperplasia in blood vessels, cutaneous scarring, and pulmonary fibrosis.

Increased levels of Hedgehog signaling are sufficient to initiate cancer formation and are required for tumor survival. These cancers include, but are not limited to, prostate cancer (“Hedgehog signalling in prostate regeneration, neoplasia and metastasis”, Karbadkar S S, Bova G S, Abdallah N, Dhara S, Gardner D, Maitra A, Isaacs J T, Berman D M, Beachy P A., Nature. 2004 Oct. 7; 431(7009):707-12; “Inhibition of prostate cancer proliferation by interference with SONIC HEDGEH0 G-GLI1 signaling”, Sanchez P, Hernandez A M, Stecca B, Kahler A J, DeGueme A M, Barrett A, Beyna M, Datta M W, Datta S, Ruiz i Altaba A., Proc Natl Acad Sci USA. 2004 Aug. 24; 101(34): 12561-6), (“Cytotoxic effects induced by a combination of cyclopamine and gefitinib, the selective hedgehog and epidermal growth factor receptor signaling inhibitors, in prostate cancer cells,” Mimeault M, Moore E, Moniaux N, et al (2006), International Journal of Cancer; 118 (4): 1022-31) breast cancer (“Hedgehog signaling pathway is a new therapeutic target for patients with breast cancer”, Kubo M, Nakamura M, Tasaki A, Yamanaka N, Nakashima H, Nomura M, Kuroki S, Katano M., Cancer Res. 2004 Sep. 1; 64(17):6071-4), (“Hedgehog signaling and Bmi-1 regulate self-renewal of normal and malignant human mammary stem cells,” Liu S, Dontu G, Mantle I D, et al (2006) Cancer Res; 66 (12):6063-71), (“Constitutive activation of smoothened (SMO) in mammary glands of transgenic mice leads to increased proliferation, altered differentiation and ductal dysplasia,” Moracs R C, Zhang X M. Harrington N, et al (2007), Development; 134 (6):1231-42), medulloblastoma (“Medulloblastoma growth inhibition by hedgehog pathway blockade”, Berman D M, Karhadkar S S, Hallahan A R, Pritchard J J, Eberhart C G, Watkins D N, Chen J K, Cooper M K, Taipale J, Olson J M, Beachy P A., Science. 2002 Aug. 30; 297(5586): 1559-61), non-melanoma skin cancer, i.e. squamous cell carcinoma (SCC) and basal cell carcinoma (BCC) (“Identification of a small molecule inhibitor of the hedgehog signaling pathway: effects on basal cell carcinoma-like lesions”, Williams J A, Guicherit O M, Zaharian B I, Xu Y, Chai L, Wichterle H, Kon C, Gatchalian C, Porter J A, Rubin L, Wang F Y., Proc Natl Acad Sci USA. 2003 Apr. 15; 100(8):4616-21; “Activating Smoothened mutations in sporadic basal-cell carcinoma”, Xie J, Murone M, Luoh S M, Ryan A, Gu Q, Zhang C, Bonifas J M, Lam C W, Hynes M, Goddard A, Rosenthal A, Epstein Eli Jr, de Sauvage F J., Nature. 1998 Jan. 1; 391(6662):90-2), pancreatic, esophagus, stomach, and biliary cancers (“Hedgehog is an early and late mediator of pancreatic cancer tumorigenesis”, Thayer S P, di Magliano M P, Heiser P W, Nielsen C M, Roberts D J, Lauwers G Y, Qi Y P, Gysin S, Fernandez-del Castillo C, Yajnik V, Antoniu B, McMahon M, Warshaw A L, Hebrok M., Nature. 2003 Oct. 23; 425(6960):851-6; “Widespread requirement for Hedgehog ligand stimulation in growth of digestive tract tumours”, Berman D M, Karhadkar S S, Maitra A, Montes De Oca R, Gerstenblith M R, Briggs K, Parker A R, Shimada Y, Eshleman J R, Watkins D N, Beachy P A., Nature. 2003 Oct. 23; 425(6960):846-51), (“Nuclear factor-kappa B contributes to hedgehog signaling pathway activation through sonic hedgehog induction in pancreatic cancer,” Nakashima H, Nakamura M, Yamaguchi H, et al (2006), Cancer Research; 66 (14):7041-9), (“Blockade of hedgehog signaling inhibits pancreatic cancer invasion and metastases: A new paradigm for combination therapy in solid cancers,” Feldmann G, Dhara S, Fendrich V, et al (2007) Cancer Research; 67 (5):2187-96), (“Oncogenic KRAS suppresses GUI degradation and activates Hedgehog signaling pathway in pancreatic cancer cells,” Ji Z, Mei F C, Xie J, et al (2007), J Biol Chem; 282 (19): 14048-55), and small-cell lung cancer (“Hedgehog signalling within airway epithelial progenitors and in small-cell lung cancer”, Watkins D N, Berman D M, Burkholder S G, Wang B, Beachy P A, Baylin S B. Nature. 2003 Mar. 20; 422(6929):313-7), (“Hedgehog signaling in small-cell lung cancer: Frequent in vivo but a rare event in vitro,” Vestergaard J, Pedersen M W, Pedersen N, et al (2006), Lung Cancer, 52 (3):281-90).

Additional cancers in which increased levels of Hedgehog signaling are sufficient to initiate cancer formation and are required for tumor survival include, but are not limited to colon cancer (“Sonic Hedgehog-dependent proliferation in a series of patients with colorectal cancer,” Douard R, Moutereau S, Pernet P, et al (2006) Surgery; 139 (5):665-70), (“Hedgehog signalling in colorectal tumour cells: Induction of apoptosis with cyclopamine treatment,” Qualtrough D, Buda A, Gaffield W, et al (2004), International Journal of Cancer, 110 (6):831-7), glioma, (“Cyclopamine-mediated Hedgehog pathway inhibition depletes stem-like cancer cells in glioblastoma,” Bar E E, Chaudhry A, Lin A, et al, Neuro-Oncology; 2007, 9 (4):594), (“HEDGEHOG-GL11 signaling regulates human glioma growth, cancer stem cell self-renewal, and tumorigenicity,” Clement V, Sanchez P, de Tribolet N, et al, (2007) Current Biology 17 (2): 165-72), (“Ligand-dependent activation of the hedgehog pathway in glioma progenitor cells,” Ehteshan M, Sarangi A, Valadez J G, et al (2007) Oncogene; Mar. 12, 2007, Epub ahead of print), melanoma (“Melanomas require HEDGEHOG-GLI signaling reaulated by interactions between GI and the RAS-MEK/AKT pathways,” Stecca B, Mas C, Clement V, et al (2007), Proceedings of the National Academy of Sciences of the United States of America; 104 (14):5895-900), non small cell lung cancer (NSCLC) (“Frequent requirement of hedgehog signaling in non-small cell lung carcinoma,” Yuan Z, Goetz J A, Singh S, et al (2007), Oncogene; 26 (7): 1046-55), ovarian, (“Hedgehog signal pathway is activated in ovarian carcinomas, correlating with cell proliferation: It's inhibition leads to growth suppression and apoptosis,” Chen X J, Horiuchi A, Kikuchi N, et al, Cancer Science; (2007) 98 (1):68-76), liver (“Activation of the hedgehog pathway in human hepatocellular carcinomas,” Huang S H, He J, Zhang X L, et al (2006), Carcinogenesis; 27 (7): 1334-40), (“Dysregulation of the Hedgehog pathway in human hepatocarcinogenesis,” Sicklick J K, Li Y X, Jayaraman A, et al (2006), Carcinogenesis; 27 (4):748-57), renal (“Clear cell sarcoma of the kidney: Up-regulation of neural markers with activation of the sonic hedgehog and Akt pathways,” Cutcliffe C, Kersey D, Huang C C, et al (2005), Clinical Cancer Research 11 (22):7986-94), Rhabdomyosarcoma, (“Rhabdomyosarcomas and radiation hypersensitivity in a mouse model of Gorlin syndrome,” Hahn H, Wojnowski L, Zimmer A M, et al (1998), Nature Medicine; 4 (5):619-22), (“Deregulation of the hedgehog signalling pathway: a possible role for the PTCH and SUFU genes in human rhabdomyoma and rhabdomyosarcoma development,” Tostar U, Malm C J, Meis-Kindblom J M, et al (2006), Journal of Pathology; 208 (1): 17-25) and Chondrosarcoma (“Constitutive hedgehog signaling in chondrosarcoma up-regulates tumor cell proliferation,” Tiet T D, Hopyan S, Nadesan P, et al (2006), American Journal of Pathology; 168 (1):321-30).

Malignant lymphoma (ML) involves the cells of the lymphatic system, and is the fifth most common cancer in the U.S. ML includes Hodgkin's disease, and non-Hodgkin's diseases which are a heterogeneous group of lymphoid proliferative diseases. Hodgkin's disease accounts for approximately 14% of all malignant lymphomas. The non-Hodgkin's lymphomas are a diverse group of malignancies that are predominately of B-cell origin. In the Working Formulation classification scheme, these lymphomas been divided into low-, intermediate-, and high-grade categories by virtue of their natural histories (see “The Non-Hodgkin's Lymphoma Pathologic Classification Project,” Cancer 49:2112-2135, 1982). The low-grade lymphomas are indolent, with a median survival of 5 to 10 years (Homing and Rosenberg, N. Engl. J. Med. 311:1471-1475, 1984). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

Multiple myeloma (MM) is a malignant tumor composed of plasma cells of the type normally found in the bone marrow. These malignant plasma cells accumulate in bone marrow and typically produce monoclonal IgG or IgA molecules. The malignant plasma cells home to and expand in the bone marrow causing anemia and immunosuppression due to loss of normal hematopoiesis. Individuals suffering from multiple myeloma often experience anemia, osteolytic lesions, renal failure, hypercalcemia, and recurrent bacterial infections. MM represents the second most common hematopoietic malignancy.

“Hedgehog related disorders,” further comprise cancers of the blood and lymphatic systems, including lymphomas, leukemia, and myelomas. The methods and combinations of the invention antagonize one or more components of the Hedgehog signaling pathway to inhibit growth and proliferation of lymphoma cells, leukemia cells, or myeloma cells. Lymphoma is malignant tumor of lymphoblasts derived from B lymphocytes. Myeloma is a malignant tumor composed of plasma cells of the type normally found in the bone marrow. Leukemia is an acute or chronic disease that involves the blood forming organs. NHLs are characterized by an abnormal increase in the number of leucocytes in the tissues of the body with or without a corresponding increase of those in the circulating blood and are classified according to the type of leucocyte most prominently involved.

In addition, it is contemplated that the combination of the present invention may be used for treating carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine and endocrine pancreatic carcinoma), esophagus, stomach, gall bladder, cervix, thyroid, and skin (including squamous cell carcinoma); tumors of the central and peripheral nervous system including astreytoma, neuroblastoma, glioma, medulloblastoma and schwannomas; tumors of mesenchymal origin including fibrosarcoma, rhabdomyosarcoma, and osteosarcoma; and other tumors including melanoma, Merkel cell carcinoma, xeroderma pigmentosum, keratoacanthoma, seminoma, thyroid follicular cancer, and teratocarcinoma. It is also contemplated that the combinations of the present invention may be used for treating mastocytosis, germ cell tumors, pediatric sarcomas, and other cancers.

Inhibitors of the kinase activity of mTOR and downstream effectors are useful in treating cancerous tumors and/or metastasis (wherever located), e.g. brain and other central nervous system tumors (eg. tumors of the meninges, brain, spinal cord, cranial nerves and other parts of central nervous system, e.g. glioblastomas or medulla blastomas); head and/or neck cancer; breast tumors; circulatory system tumors (e.g. heart, mediastinum and pleura, and other intrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, rectosigmoid junction, rectum, anus and anal canal), tumors involving the liver and intrahepatic bile ducts, gall bladder, other and unspecified parts of biliary tract, pancreas, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervix uteri, Corpus uteri, uterus, ovary, and other sites associated with female genital organs, placenta, penis, prostate, testis, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

The therapeutic methods described herein may be used in combination with other cancer therapies. For example, Hh antagonists in combination with kinase inhibitors of mTOR may be administered adjunctively with any of the treatment modalities, such as chemotherapy, radiation, and/or surgery. For example, they can be used in combination with one or more chemotherapeutic or immunotherapeutic agents; and may be used after other regimen(s) of treatment is concluded. Examples of chemotherapeutic agents which may be used in the compositions and methods of the invention include but are not limited to anthracyclines, alkylating agents (e.g., mitomycin C), alkyl sulfonates, aziridines, ethylenimines, methylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins.

Particular examples of known chemotherapeutic agents which may be used in the compositions and methods of the invention include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipabroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidinc, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elfornithine, elliptinium acetate, etoglucid, etoposide, flutamide, gallium nitrate, hydroxyurea, interferon-alpha, interferon-beta, interferon-gamma, interleukin-2, lentinan, lonidamine, prednisone, dexamethasone, leucovorin, mitoguazone, mitoxantrone, mopidamol, nitracrine, pentostatin, phenamet, pirarubicin, podophyllinic acid, 2-ethylhydrazide, procatazine, razoxane, sizofiran, spirogermanium, paclitaxel, tamoxifen, teniposide, tenuazonic acid, triaziquone, 2,2′,2″-trichlotriethylamine, urethane, vinblastine, vincristine, and vindesine.

In accordance with the foregoing, the present invention further provides a method for treating any of the diseases or disorders described above in a subject in need of such treatment, which method comprises administering to said subject a therapeutically effective amount (See, “Administration and Pharmaceutical Compositions”, infra) of the pharmaceutically active agents or pharmaceutically acceptable salt thereof. For any of the above uses, the required dosage will vary depending on the mode of administration, the particular condition to be treated and the effect desired.

Administration and Pharmaceutical Compositions:

In general, compounds of the invention will be administered in therapeutically effective amounts via any of the usual and acceptable modes known in the art, either singly or in combination with one or more therapeutic agents. A combination of the present invention includes administration at the same time as well as sequential administration. A therapeutically effective amount may vary widely depending on the severity of the disease, the age and relative health of the subject, the potency of the compound used and other factors. An indicated daily dosage in the larger mammal, e.g. humans, is in the range from about 5 mg to about 2,500 mg, more preferably about 100 mg to 3000 mg, in dosages such as 100 mg, 200 mg, 400 mg, 500 mg, 600 mg, 700 mg, 800 mg, 900 mg and 1000 mg. These dosages can be conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with immunomodulatory or anti-inflammatory substances or other anti-tumor therapeutic agents. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

One of skill in the art will appreciate that the above transformations are only representative of methods for preparation of the compounds of the present invention, and that other well known methods can similarly be used.

### Example 1

### Description of Subcutaneous Medulloblastoma Allograft Models

Mouse medulloblastoma cells (1.0-5.0×10⁶), dissociated directly from tumor fragments originally derived from spontaneously arising medulloblastomas in Ptch+/−Hic+/− mice, were inoculated subcutaneously into the right flank of Harlan nu/nu mice. Treatment was initiated approximately 7-10 days post implantation. Animals were randomized into treatment groups with similar mean tumor volumes that ranged from approximately 250-300 mm3. Tumor volumes (mm3) and body weights (g) were recorded two or three times per week from all groups for analysis. Dose was body weight adjusted at time of dosing. Comparisons between treatment groups was performed using a non-parametric Kruskal-Wallis/Wilcoxon Rank Sum Test.

### Example 2

### Allograft Model Data Analysis

Tumors were calipered in two dimensions, and the volumes were calculated using the formula: (length×width2)/2, where length is the longer of the two measurements and width is the shorter one. Percent treatment/control (% T/C) values were calculated using the following formula: % T/C=100×ΔTf-i/ΔCf-i if ΔTf-i>0, % T/T0-100×ΔTf-i/T0 if ΔTf-i<0 (regression). A partial responder (PR) was defined as an animal whose tumor 3$ was less than 50% of the initial tumor volume by the end of the study. An animal with no palpable tumor by the end of study is defined as a complete responder (CR).

Ptch+/− mice develop medulloblastoma spontaneously (Romer, et al., Cancer Cell, Volume 6, Issue 3, pages 229-24, 2004). The tumors, which have been previously shown to be Smo-dependent, are used as models to test compounds which inhibit the Hh pathway. The heterozygous loss of Hic results in an earlier onset and increases the incidence rate of medulloblastomas (Briggs et al., Genes & Dev., 22: pages 770-785 2008). The in vivo efficacy of Compound 1 was evaluated in Ptch+/−Hic+/− mouse medulloblastoma allograft models, derived from corresponding transgenic mice and passaged in vivo, and following long-term continuous dosing. The following experiments were performed to evaluate if mTor inhibitors could be used either alone or in combination to treat resistant tumors or to overcome the development of resistance to Smo antagonists.

### Example 3

### Treatment with mTor Inhibitors and Smo Antagonists

Compound A, also know as everolimus, an allosteric inhibitor of mTor (a downstream signaling molecule in the PI3K pathway), was used to evaluate the role of the PI3 kinase pathway in medulloblastoma.

The effect of mTor inhibitors (e.g., compounds such as Compound A) on the proliferation of medulloblastoma cells derived from sensitive and resistant medulloblastoma tumors was evaluated by using an “ex-vivo medulloblastoma proliferation assay. Sensitive tumors means tumors (e.g., medulloblastomas) which, due to hedgehog pathway activation, respond to treatment with a smoothened inhibitor anti-cancer regimen. Resistant tumors means formerly sensitive tumors (e.g., medulloblastomas) which, in the continuos presence of a smo inhibitor, either have regrown after shrinking due to treatment, or have reappeared after being temporarily eliminated due to treatment. Resistant tumors show a decreased sensitivity or no response to smoothened inhibition.

Using Ptch+/−Hic+/− medulloblastoma tumors freshly harvested from allografted nude mice, we have developed a short-term 48 h proliferation assay that enables us to assess the in vitro potency of Smo inhibitors. The read-out for proliferation uses incorporation of 3H thymidine. The assay reflects the in vivo sensitivity of tumor cells to Compound 1.

Table 1 summarizes the results of treating medulloblastoma cells in culture with Compound 1 (Smo inhibitor) and Compound A (mTor inhibitor), or combinations thereof. As shown in Table 1, sensitive cells were inhibited by compound 1 with an IC50 of 8 nM whereas the IC50 was 9 μM in resistant tumors. However, the mTor inhibitor Compound A inhibited both sensitive and resistant tumors with similar IC50s. The potency of compound A in resistant tumors was increased in the presence of 5 and 20 uM of compound 1.

TABLE 1

| Tumor | Compound | Compound | Compound 1 + A |
|---|---|---|---|
| type | 1 (uM) | A(uM) | (uM) |
| Sensitive | 0.008 | 0.009 | 0.002 |
| Resistant | 9.08 | 0.002 | 0.0002 |
| | | | (5 uM 225) |
| | | | <7.62e−005 |
| | | | (20 uM 225) |

### Example 4

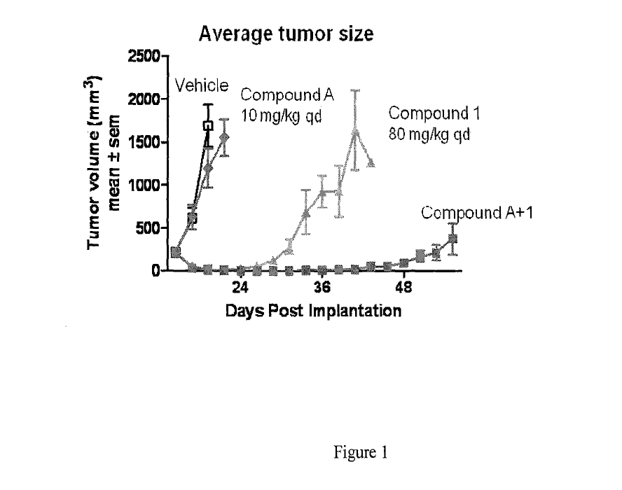

Next, a combination of Compound 1 and Compound A was explored in the Ptch+/−Hic+/− medulloblastoma allograft model. As shown in FIG. 1, tumor-bearing animals were dosed po with 80 mg/kg qd of Compound 1, 10 mg/kg qd of Compound A, and a combination of Compound 1 and Compound A. Whereas Compound A had only a moderate effect on tumor growth compared to vehicle control, Compound 1 initially induced regression but tumors started to regrow. Tumor regrowth in animals treated with the combination of Compound 1 and Compound B was considerably delayed.

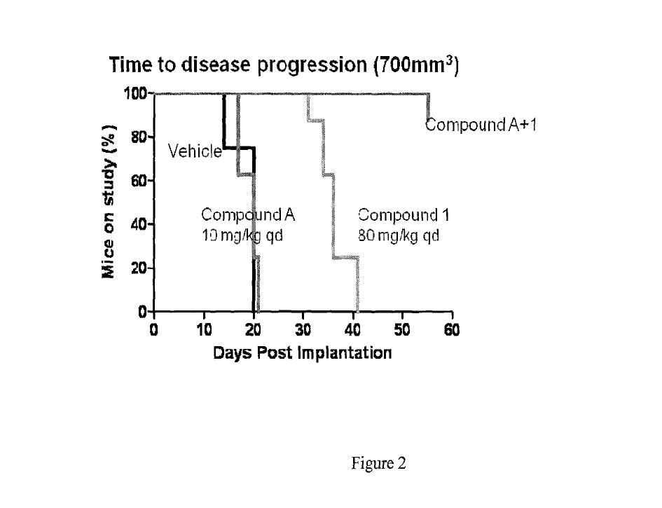

The combination treatment resulted in prolonged time to endpoint (tumor volume>700 mm3), as shown in FIG. 2. Animals treated with vehicle control and with compound A reached the endpoint around day 20 because their tumor volume reached 700 mm3. The time to endpoint was significantly prolonged in animals treated with Compound 1. In the combination treatment group the majority of mice remained on study can significantly delay or prevent the development of resistance in the medulloblastoma model.

1: A method of treating a cancer tumor in a patient in need thereof comprising administering to the patient a first agent that is a Smoothened inhibitor and a second agent that is an mTOR inhibitor, wherein the first agent is 2-methyl-4′-trifluoromethoxy-biphenyl-3-carboxylic acid [6-(cis-2,6-dimethyl-morpholin-4-yl)-pyridin-3-yl]-amide or a pharmaceutically acceptable salt thereof and wherein the second agent is everolimus, wherein the cancer tumor has hedgehog pathway activation and is responsive to treatment with the Smoothened inhibitor.

2: The method of claim 1, wherein the cancer tumor is medulloblastoma.
